SEQUENCE LISTING (24 sequences)

SEQ ID NO: 1 - DNA, 2010 bp, Arabidopsis thaliana
atggcttcgtacactcccaagaacattctcatcaccggagctgctggtttcattgcgtctcatgtcgccaacagactcatacgaagctatcctgattacaaaatcgttgtgcttgacaagcttgattactgttcaaatctcaagaatctcaatccttctaagcactctccgaacttcaagtttgtcaaaggtgatatcgctagtgctgacttggtgaatcatcttctcatcactgaaggtattgacaccatcatgcatttcgctgctcagactcacgtcgacaattccttcggtaacagtttcgagtttactaagaataatatctatggaactcatgtccttcttgaggcttgtaaagttactggtcagattaggaggtttattcatgttagtactgatgaagtttatggtgaaactgatgaggatgctcttgttggtaaccatgaggcttctcagctgcttccgacgaatccttactctgccacgaaagctggtgctgagatgcttgttatggcttatggtagatcttatggtttgcctgttattaccactcgtgggaataacgtctatggaccgaatcagtttcctgagaagttgattcctaagttcattttgctggcaatgagagggcaggttcttcccattcatggagatggatcaaatgtcaggagctacctctactgtgaagacgttgctgaggcttttgaagttgttcttcacaagggagaagttggccatgtttacaatattgggacgaagaaggagaggagagtgaatgatgttgccaaagacatctgcaaactcttcaacatggaccctgaggcgaacatcaagtttgtcgacaacagaccttttaacgatcagaggtacttccttgacgatcagaagctcaaaaagttgggatggtcagagagaaccacgtgggaagaagggttgaagaaaactatggattggtacacacagaacccggagtggtggggtgatgtttctggagcattgcttcctcatccaaggatgctgatgatgcctggtgggcgacactttgatggctccgaggacaattcgctggcagctactttatctgaaaaaccaagtcaaacccatatggttgttccaagccaaaggagcaacggcacacctcaaaagccttcgctgaagttcctgatatatggaaagaccggatggatcggtggtctgcttggaaagatatgtgataagcaaggaattgcttacgagtatgggaaaggtcggttggaggatcgatcttctcttctgcaggatattcagagtgttaagccaacccatgttttcaattccgctggtgtgactgggagacccaatgttgactggtgtgagtctcacaagaccgagactatccgtgccaatgtagctggcacattgactctagctgatgtctgcagagagcacggactcctaatgatgaatttcgctactggttgtatattcgaatatgacgacaagcatccggaaggttcaggaattggcttcaaggaggaagacacacccaacttcactggctctttctactcgaaaaccaaagccatggtcgaggagctgctaaaggagtatgacaacgtatgcacattgagggtaaggatgccgatctcctcggatctaaacaacccgcgcaacttcatcaccaagatctccaggtacaacaaagtagtgaacatcccaaacagcatgactgtgttggacgagttattaccaatctccatcgagatggcgaaaagaaacttgaaaggaatctggaacttcacaaacccaggtgtggtgagccacaacgagatcctagagatgtacagagactacatcaaccctgaattcaaatgggcaaacttcacattagaggagcaagctaaagtcattgtggctccaagaagcaacaacgagatggatgcttccaagctcaagaaagagttccctgagctactctctatcaaggagtctctgattaagtatgcatacgggccaaacaagaaaacctga

SEQ ID NO: 2 - protein, 669 aa, Arabidopsis thaliana
MASYTPKNILITGAAGFIASHVANRLIRSYPDYKIVVLDKLDYCSNLKNLNPSKHSPNFKFVKGDIASADLVNHLLITEGIDTIMHFAAQTHVDNSFGNSFEFTKNNIYGTHVLLEACKVTGQIRRFIHVSTDEVYGETDEDALVGNHEASQLLPTNPYSATKAGAEMLVMAYGRSYGLPVITTRGNNVYGPNQFPEKLIPKFILLAMRGQVLPIHGDGSNVRSYLYCEDVAEAFEVVLHKGEVGHVYNIGTKKERRVNDVAKDICKLFNMDPEANIKFVDNRPFNDQRYFLDDQKLKKLGWSERTTWEEGLKKTMDWYTQNPEWWGDVSGALLPHPRMLMMPGGRHFDGSEDNSLAATLSEKPSQTHMVVPSQRSNGTPQKPSLKFLIYGKTGWIGGLLGKICDKQGIAYEYGKGRLEDRSSLLQDIQSVKPTHVFNSAGVTGRPNVDWCESHKTETIRANVAGTLTLADVCREHGLLMMNFATGCIFEYDDKHPEGSGIGFKEEDTPNFTGSFYSKTKAMVEELLKEYDNVCTLRVRMPISSDLNNPRNFITKISRYNKVVNIPNSMTVLDELLPISIEMAKRNLKGIWNFTNPGVVSHNEILEMYRDYINPEFKWANFTLEEQAKVIVAPRSNNEMDASKLKKEFPELLSIKESLIKYAYGPNKKT

SEQ ID NO: 3 - DNA, 2004 bp, Arabidopsis thaliana
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 4 - protein, 667 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 5 - DNA, 1143 bp, Arabidopsis thaliana
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagct

SEQ ID NO: 6 - protein, 381 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKA

SEQ ID NO: 7 - DNA, 894 bp, Arabidopsis thaliana
acacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 8 - protein, 297 aa, Arabidopsis thaliana
TPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 9 - DNA, 1167 bp, Arabidopsis thaliana
atgcatcaccatcaccatcacatggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttaa

SEQ ID NO: 10 - protein, 388 aa, Arabidopsis thaliana
MHHHHHHMDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKA

SEQ ID NO: 11 - DNA, 915 bp, Arabidopsis thaliana
atgcatcaccatcaccatcacacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 12 - protein, 304 aa, Arabidopsis thaliana
MHHHHHHTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 13 - DNA, 53 bp, Artificial Sequence
  Chemically synthesized primer for RHM1
aagagctcatgcatcaccatcaccatcacatggcttcgtacactcccaagaac

SEQ ID NO: 14 - DNA, 35 bp, Artificial Sequence
  Chemically synthesized primer for RHM1
aaaggtacctcaggttttcttgtttggcccgtatg

SEQ ID NO: 15 - DNA, 58 bp, Artificial Sequence
  Chemically synthesized primer for RHM2
agaattcatgcatcaccatcaccatcacatggatgatactacgtataagccaaagaac

SEQ ID NO: 16 - DNA, 38 bp, Artificial Sequence
  Chemically synthesized primer for RHM2
aaaaagtcgacttaggttctcttgtttggttcaaagac

SEQ ID NO: 17 - DNA, 38 bp, Artificial Sequence
  Chemically synthesized primer for RHM2-N
aaaaagtcgacttaagctttgtcaccagaatcaccatt

SEQ ID NO: 18 - DNA, 53 bp, Artificial Sequence
  Chemically synthesized primer for RHM2-C
agaattcatgcatcaccatcaccatcacacacctaagaatggtgattctggtg

SEQ ID NO: 19 - DNA, 1995 bp, Arabidopsis thaliana
atggctacatataagcctaagaacatcctcatcactggggctgctggattcatagcctctcatgttgctaacagactagttcgcagctaccctgactacaaaattgttgtgcttgacaagcttgattactgttctaatctgaagaaccttaatccttctaaatcctctcccaacttcaagtttgtgaaaggagatatcgccagtgctgatctcgtcaactaccttctcatcactgaagaaatcgacaccattatgcactttgctgctcaaacccatgttgacaattctttcggtaatagctttgagtttaccaagaacaatatttatggtacccatgtccttttggaagcttgtaaagtcactggccagatcaggaggttcatccatgtgagtactgatgaggtctatggagagactgatgaggatgcttcagtgggtaatcacgaggcttctcagttgctcccaactaatccatactccgccactaaagctggagctgagatgcttgtcatggcatatggtagatcatatgggttgccggttataacaactcgcgggaacaatgtttatggtcctaaccagtttcctgaaaagttgattcctaagttcatcctcttggccatgaatgggaagcctctcccaatccacggagatggatctaatgtgagaagttatctctactgcgaagatgttgctgaggcatttgaggttgttcttcacaaaggggaagttaaccatgtctacaatatagggacaacgagagaaaggagagtgattgatgtggctaatgacatcagtaaactctttgggatagaccctgactccaccattcagtatgtggaaaaccggccattcaatgaccagaggtacttcctcgatgaccagaagctgaagaaattaggatggtgtgagcgaaccaattgggaagaaggactgaggaagacaatggaatggtatactgagaaccctgagtggtggggcgatgtttctggagctctgcttcctcatccacggatgttgatgatgcccggtgaccgacactctgatggctctgacgagcacaagaatgcagatggtaatcagacattcacggtggttactcccaccaaggctggttgttccggagacaaaagatccttgaagttcctcatctatgggaagactgggtggctcggtggtcttctgggaaaactatgtgagaaacaagggattccttacgagtatggaaaaggaagactagaggatagagcttctctcatcgcagatattcgcagcatcaaaccaagtcatgtcttcaacgccgctggtttaactgggagacccaatgttgactggtgtgaatctcacaaaactgaaaccatccgagtcaacgttgctggaactttgactcttgcagatgtttgcagagagaatgatctgttgatgatgaactttgccactggttgtatattcgagtatgacgctgcacatccagaaggttcagggattggttttaaggaagaagataaaccgaatttcactggttctttctactcaaaaacaaaggcaatggtggaagagcttctaagagaatttgacaacgtatgcaccttgagagtgcggatgccaatctcatctgacttaaataacccgcgaaacttcatcacgaagatctcgcgttacaacaaagtggtgaacattccaaacagcatgaccatactagatgaactcttaccaatctcgatcgagatggcgaagaggaacctaaggggaatatggaacttcaccaatccaggagtggtgagccacaacgagatattagagatgtacaagagttacatcgagcctgatttcaaatggtccaacttcaatttggaagaacaggctaaggtcattgttgctccacggagcaacaacgagatggatggtgccaagctcagcaaggagtttccagagatgctttccatcaaagattcgttgatcaaatacgtcttcgaacctaacaagagaacgtaa

SEQ ID NO: 20 - protein, 664 aa, Arabidopsis thaliana
MATYKPKNILITGAAGFIASHVANRLVRSYPDYKIVVLDKLDYCSNLKNLNPSKSSPNFKFVKGDIASADLVNYLLITEEIDTIMHFAAQTHVDNSFGNSFEFTKNNIYGTHVLLEACKVTGQIRRFIHVSTDEVYGETDEDASVGNHEASQLLPTNPYSATKAGAEMLVMAYGRSYGLPVITTRGNNVYGPNQFPEKLIPKFILLAMNGKPLPIHGDGSNVRSYLYCEDVAEAFEVVLHKGEVNHVYNIGTTRERRVIDVANDISKLFGIDPDSTIQYVENRPFNDQRYFLDDQKLKKLGWCERTNWEEGLRKTMEWYTENPEWWGDVSGALLPHPRMLMMPGDRHSDGSDEHKNADGNQTFTVVTPTKAGCSGDKRSLKFLIYGKTGWLGGLLGKLCEKQGIPYEYGKGRLEDRASLIADIRSIKPSHVFNAAGLTGRPNVDWCESHKTETIRVNVAGTLTLADVCRENDLLMMNFATGCIFEYDAAHPEGSGIGFKEEDKPNFTGSFYSKTKAMVEELLREFDNVCTLRVRMPISSDLNNPRNFITKISRYNKVVNIPNSMTILDELLPISIEMAKRNLRGIWNFTNPGVVSHNEILEMYKSYIEPDFKWSNFNLEEQAKVIVAPRSNNEMDGAKLSKEFPEMLSIKDSLIKYVFEPNKRT

SEQ ID NO: 21 - DNA, 2028 bp, Oryza sativa
atggcggcttacgagcccaagaacattcttataaccggcgctgctggtttcatcgcatcacatgtggcgaaccgcctagtcaggaactatccgcactacaagattgttgtcctcgacaagctcgattactgttccagcctgagcaatctcaacccttcacgtccatcgccgaatttcaagtttgtcaagggtgacattgcaagtgctgatctggtgaactaccttctcacaactgagtcaattgacactatcatgcactttgctgctcagactcatgtggataattcatttggcaattcctttgagtttacaaagaacaatatttatggtacccatgtccttcttgaggcctgcaaggttactggtcagattagaagatttattcatgtgagtactgacgaggtgtacggagaaactgatgaggatgctgtggttggcaaccatgaggcctcacagctgcttccaacaaacccatattcagctacaaaagctggggctgagatgcttgtgatggcttatggaagatcttatggccttcctgtgattacaactcggggcaacaatgtatatgggccaaatcagttccctgagaagctcattcccaaatttattcttttggcaatgagaggcttgccccttcccattcacggtgatggctcaaatgtcagaagctatctgtattgtgaggatgttgctgaagcttttgaggtggttcttcacaaaggagaggttgggcatgtgtacaatattggtactgtgaaggaaaggagggtgattgatgtggccaaggacatatgcaagctctttggcttggacaccgagaaggtcatcagatttgttgagaacaggcctttcaacgaccagagatacttcttggatgatcagaagctgaagaaattgcgatgggcagagcgcacactatgggaagaggggttgaagaaaacaattgaatggtacactaacaatcctgattactggggagatgtcgcaggtgccttgctccctcatccaaggatgttgatgacacctggagttgaaaggcataactggactgatgagatcaaatctctttccacttcaccagatgaagctaaggaatctagcactgcggttcctgcagccactgccaaaagcaccagcagtgcccctcaaaaggcctcatacaagttcttgatatatggtaggactggatggattggtggtctacttggtaagatatgtgagaagcaagggattccatatgagtatgggaaaggacgcctggaagagcgctctcagctcttgcaggacattagaaatgtgaagccaactcatgttttcaatgctgctggtgtcactgggaggccaaatgttgactggtgcgagacccataaacaggatactatccgcaccaatgttgttggcaccttgaaccttgctgatgtttgtcgtgagcagggcttgctcatgattaattatgctacaggttgcatatttgagtatgatgctaagcaccccgaaggatcaggaattggttttaaagaggaagataagcccaactttactggttcatattattccaaaactaaagctatggttgaagagctgttgcaagaatatgacaacgtctgtacgcttcgagtcaggatgcccatatcatcagatttgagcaacccccgtaacttcatcacaaagatagctcgttatgacaaggtggtaatcatccccaacagtatgacaattttggatgagcttttgcccatctccattgagatggcgaagcgggactgcaggggcatatggaacttcaccaatcctggggttgtcagccacaacgagatcctggagatgtacaagaaatacctcaaccccgacttcaagtggaccaacttcactcttgaggaacaggccaaggttatagttgcaccaagaagtaacaacgaaatggatgcatcgaagttgaagtccgagttccccgagctactgtccatcaaggattctttggttaagtatgtttttgagccaaacaggaaggttcctgctaactga

SEQ ID NO: 22 - protein, 675 aa, Oryza sativa
MAAYEPKNILITGAAGFIASHVANRLVRNYPHYKIVVLDKLDYCSSLSNLNPSRPSPNFKFVKGDIASADLVNYLLTTESIDTIMHFAAQTHVDNSFGNSFEFTKNNIYGTHVLLEACKVTGQIRRFIHVSTDEVYGETDEDAVVGNHEASQLLPTNPYSATKAGAEMLVMAYGRSYGLPVITTRGNNVYGPNQFPEKLIPKFILLAMRGLPLPIHGDGSNVRSYLYCEDVAEAFEVVLHKGEVGHVYNIGTVKERRVIDVAKDICKLFGLDTEKVIRFVENRPFNDQRYFLDDQKLKKLRWAERTLWEEGLKKTIEWYTNNPDYWGDVAGALLPHPRMLMTPGVERHNWTDEIKSLSTSPDEAKESSTAVPAATAKSTSSAPQKASYKFLIYGRTGWIGGLLGKICEKQGIPYEYGKGRLEERSQLLQDIRNVKPTHVFNAAGVTGRPNVDWCETHKQDTIRTNVVGTLNLADVCREQGLLMINYATGCIFEYDAKHPEGSGIGFKEEDKPNFTGSYYSKTKAMVEELLQEYDNVCTLRVRMPISSDLSNPRNFITKIARYDKVVIIPNSMTILDELLPISIEMAKRDCRGIWNFTNPGVVSHNEILEMYKKYLNPDFKWTNFTLEEQAKVIVAPRSNNEMDASKLKSEFPELLSIKDSLVKYVFEPNRKVPAN

SEQ ID NO: 23 - DNA, 58 bp, Artificial Sequence
  Chemically synthesized primer for RHM3
agaattcatgcatcaccatcaccatcacatggctacatataagcctaagaacatcctc

SEQ ID NO: 24 - DNA, 39 bp, Artificial Sequence
  Chemically synthesized primer for RHM3
aaaaagtcgacttacgttctcttgttaggttcgaagacg

(19) United States
(12) Patent Application Publication
(10) Pub. No.: US 2008/0064069 A1
(43) Pub. Date: Mar. 13, 2008

(54) Method for producing UDP-rhamnose and enzyme used for the method

(75) Inventors: Takuji Oka, Tsukuba-shi (JP); Yoshifumi Jigami, Tsukuba-Shi (JP)

Correspondence Address: BIRCH STEWART KOLASCH & BIRCH, PO BOX 747, FALLS CHURCH, VA, 22040-0747, US

(73) Assignee: National Institute of Advanced Industrial Science and Technology, Tokyo (JP)

(21) Appl. No.: 11/802,375

(22) Filed: May 22, 2007

(30) Foreign Application Priority Data
May 23, 2006 (JP) 2006-142359
Jul. 27, 2006 (JP) 2006-204421

(51) Int. Cl. C07H 21/04; C12N 15/00; C12N 5/04; C12N 5/08; C12P 19/00

(52) U.S. Cl. 435/72; 435/192; 435/232; 435/255.1; 435/320.1; 536/23.2

(57) ABSTRACT

A protein having (a) an amino acid sequence represented by SEQ ID NO: 2, SEQ ID NO: 4, SEQ ID NO: 20, SEQ ID NO: 6 or SEQ ID NO: 8, or (b) an amino acid sequence represented by SEQ ID NO: 2, SEQ ID NO: 4, SEQ ID NO: 20, SEQ ID NO: 6 or SEQ ID NO: 8, wherein one or more amino acids are deleted, substituted and/or added in the amino acid sequence set forth in SEQ ID NO: 2, SEQ ID NO: 4, SEQ ID NO: 20, SEQ ID NO: 6 or SEQ ID NO: 8, and said sequence has a UDP-glucose 4,6-dehydratase activity, and/or UDP-4-keto-6-deoxyglucose 3,5-epimerase and UDP-4-keto-rhamnose 4-keto-reductase activities; and a method of producing UDP-rhamnose using the above-described protein.

### FIELD OF THE INVENTION

The present invention relates to a gene involved in synthesis of UDP-rhamnose, a recombinant vector having the gene, a transformant transformed with the recombinant vector, and a method of producing UDP-rhamnose by using the transformant.

### BACKGROUND OF THE INVENTION

It is revealed that sugar chains of glycoproteins and the like play a very important role in the living body, and consequently, glycoengineering for arbitrarily modifying a sugar-chain structure becomes an important technology. At present, the technique of modifying a sugar chain includes a chemical method in which a chemically synthesized objective sugar chain is bound to a protein; and a biological method in which a sugar chain is converted into an objective sugar chain by using a genetic engineering method of using an intrinsic function of cells to synthesize a sugar chain or by reacting a glycosyltransferase itself with a protein thereby giving an objective glycoprotein. The chemical method opens the door leading to synthesis of an objective sugar chain in a large amount, but, due to the complexity of the sugar chain, does not arrive at easy supply of every kind of sugar chain or fails to bind a large amount of a chemically synthesized sugar chain efficiently to a protein. In the biological method, on the other hand, expression of a sugar chain synthesis-related gene and control of the function thereof are made possible by development of genetic engineering, thus enabling in vivo modification of a sugar chain by using living cells in the living body. Further, synthesis of a sugar chain by using a glycosyltransferase in vitro is also very useful and enables production of a large amount of uniform sugar chains. In the in vivo and in vitro synthesis of a sugar chain by using the biological method, on the other hand, a sugar nucleotide is essential as a glycosyl donor (saccharide donor) for the glycosyltransferase, and this sugar nucleotide is too expensive to be used in mass production. This is because the sugar nucleotide is a labile and highly reactive substance bound via a high-energy bond and it is produced in a very small amount in the living body, thus making its production amount low in living things and allowing the sugar chain to be hardly produced in a large amount.

In recent years, a system of producing relatively many kinds of sugar nucleotides is increasingly practically applicable by using a system of production using bacteria, thus enabling sugar nucleotides to be supplied more stably. However, in this system of using bacteria, sugar nucleotides are produced by mixing two kinds of bacteria and disrupting the bacteria in order to send raw materials contained in cells in one kind of bacteria to those in the other kind of bacteria, so the amount of sugar nucleotides produced is not so high where the reaction process is long to some extent, and thus there is demand for development of new methods. In addition, there is no report on a system for mass production of UDP-rhamnose in a production system by using bacteria.

Among sugar nucleotides, UDP-rhamnose is essential for synthesis of a rhamnose-containing sugar chain as the glycosyl donor for a rhamnose transferase. Many of the rhamnose-containing sugar chains represented by rhamnogalacturonan I (RG-I) or rhamnoglacturonan II (RG-II) fulfill a functionally important role, and thus it is desired to supply the glycosyl donor inexpensively in a large amount. Further, there are also rhamnose-containing O-linked sugar chains, and the UDP-rhamnose is also used as the glycosyl donor for biosynthesis of such sugar chains. Accordingly, it is estimated that as the research and development of these rhamnose-containing substances are advanced to reveal their important functions, new demand therefor is increased. This UDP-rhamnose is synthesized from UDP-glucose in a 3-stage reaction catalyzed by one enzyme (RHM2) (FIG. 1). The enzyme catalyzing this 3-stage reaction is encoded by a single gene whose N-terminal domain and C-terminal domain are different from each other in function. That is, the first-stage reaction is caused by UDP-glucose 4,6-dehydratase encoded in the N-terminal domain of the RHM2 protein, to give UDP-4-keto-6-deoxyglucose. The subsequent second- and third-stage reactions are caused by UDP-4-keto-6-deoxyglucose 3,5-epimerase and UDP-4-keto-rhamnose 4-keto-reductase, respectively, encoded in the C-terminal domain of the RHM2 protein, to give UDP-rhamnose.

These enzymes are universal enzymes possessed by any living things using the UDP-rhamnose. However, such living things consume synthesized UDP-rhamnose, therefore the UDP-rhamnose will not be accumulated in their cells. Accordingly, when the UDP-rhamnose is isolated from living things, the amount of UDP-rhamnose produced is very low and the UDP-rhamnose is expensive. Further, there is no example of synthesis of the UDP-rhamnose by genetic engineering means.

### SUMMARY OF THE INVENTION

The present invention resides in a protein having (a) an amino acid sequence represented by SEQ ID NO: 2, SEQ ID NO: 4, SEQ ID NO: 20, SEQ ID NO: 6 or SEQ ID NO: 8, or (b) an amino acid sequence represented by SEQ ID NO: 2, SEQ ID NO: 4, SEQ ID NO: 20, SEQ ID NO: 6 or SEQ ID NO: 8, wherein one or more amino acids are deleted, substituted and/or added in the amino acid sequence set forth in SEQ ID NO: 2, SEQ ID NO: 4, SEQ ID NO: 20, SEQ ID NO: 6 or SEQ ID NO: 8, and said sequence has a UDP-glucose 4,6-dehydratase activity, and/or UDP-4-keto-6-deoxyglucose 3,5-epimerase and UDP-4-keto-rhamnose 4-keto-reductase activities.

Further, the present invention resides in a method of producing UDP-rhamnose using the above-described protein.

Other and further features and advantages of the invention will appear more fully from the following description, appropriately referring to the accompanying drawings.

### BRIEF DESCRIPTION OF THE DRAWINGS

FIG. 1 is a view showing the outline of a process for synthesis of UDP-rhamnose.

FIG. 2 is a photograph in which expression of RHM1, RHM2 and RHM3 proteins by the transformants obtained in Example 2 was confirmed by Western blotting.

FIG. 3 is profiles showing the result of UDP-rhamnose synthesis using the cytoplasmic fraction from each of the transformants obtained in Example 5.

FIG. 4 is a profile showing the result of measurement, by ESI-MS, of a fraction separated by HPLC in Example 5.

FIG. 5 is a profile showing the result of HPLC detection of UDP-rhamnose separated from the cytoplasm in Example 6.

FIG. 6 is a photograph in which expression of RHM2-N and RHM2-C proteins by the transformants obtained in Example 7 was confirmed by Western blotting.

FIG. 7 is profiles showing the result of UDP-rhamnose synthesis using the cytoplasmic fraction from each of the transformants obtained in Example 10.

### DETAILED DESCRIPTION OF THE INVENTION

The present inventors made extensive study for solving the problems described above, and as a result, they first isolated a gene encoding UDP-glucose 4,6-dehydratase, UDP-4-keto-6-deoxyglucose 3,5-epimerase and UDP-4-keto-rhamnose 4-keto-reductase (hereinafter also referred to as UDP-glucose 4,6-dehydratase-3,5-epimerase/4-keto reductase) catalyzing synthesis of UDP-rhamnose in Arabidopsis (Arabidopsis thaliana), and revealed the structure of this gene. In addition, they found that by expressing this gene functionally, the UDP-rhamnose can be synthesized efficiently in vivo and in vitro. Further, the present inventors surprisingly found that when the above gene is divided into the UDP-glucose 4,6-dehydratase domain and the UDP-4-keto-6-deoxyglucose 3,5-epimerase and UDP-4-keto-rhamnose 4-keto-reductase domain and the both domains are co-expressed (separately expressed), the efficiency of production of the UDP-rhamnose is improved as compared with the case where the gene containing all these domains is expressed. The present invention was thereby attained based on these findings.

According to the present invention, there is provided the following means:

(1) A protein comprising:

(a) an amino acid sequence represented by SEQ ID NO: 2, SEQ ID NO: 4 or SEQ ID NO: 20, or

(b) an amino acid sequence represented by SEQ ID NO: 2, SEQ ID NO: 4 or SEQ ID NO: 20, wherein one or more amino acids are deleted, substituted and/or added in the amino acid sequence set forth in SEQ ID NO: 2, SEQ ID NO: 4 or SEQ ID NO: 20, and said sequence has UDP-glucose 4,6-dehydratase, UDP-4-keto-6-deoxyglucose 3,5-epimerase and UDP-4-keto-rhamnose 4-keto-reductase activities;

(2) A DNA, which encodes the protein as described in the above item (1);

(3) A DNA comprising:

(a) a nucleotide sequence represented by SEQ ID NO: 1, SEQ ID NO: 3 or SEQ ID NO: 19, or

(b) a nucleotide sequence represented by SEQ ID NO: 1, SEQ ID NO: 3 or SEQ ID NO: 19, wherein one or more nucleotides are deleted, substituted and/or added in the nucleotide sequence set forth in SEQ ID NO: 1, SEQ ID NO: 3 or SEQ ID NO: 19, and said sequence encodes a protein having UDP-glucose 4,6-dehydratase, UDP-4-keto-6-deoxyglucose 3,5-epimerase and UDP-4-keto-rhamnose 4-keto-reductase activities;

(4) A recombinant vector comprising the DNA as described in the above item (2) or (3);

(5) A transformant, which is obtained by introducing the recombinant vector as described in the above item (4) into a host cell;

(6) A protein comprising:

(a) an amino acid sequence represented by SEQ ID NO: 6, or

(b) an amino acid sequence represented by SEQ ID NO: 6, wherein one or more amino acids are deleted, substituted and/or added in the amino acid sequence set forth in SEQ ID NO: 6, and said sequence has a UDP-glucose 4,6-dehydratase activity;

(7) A DNA, which encodes the protein as described in the above item (6);

(8) A DNA comprising:

(a) a nucleotide sequence represented by SEQ ID NO: 5, or

(b) a nucleotide sequence represented by SEQ ID NO: 5, wherein one or more nucleotides are deleted, substituted and/or added in the nucleotide sequence set forth in SEQ ID NO: 5, and said sequence encodes a protein having a UDP-glucose 4,6-dehydratase activity;

(9) A recombinant vector comprising the DNA as described in the above item (7) or (8);

(10) A transformant, which is obtained by introducing the recombinant vector as described in the above item (9) into a host cell;

(11) A protein comprising:

(a) an amino acid sequence represented by SEQ ID NO: 8, or

(b) an amino acid sequence represented by SEQ ID NO: 8, wherein one or more amino acids are deleted, substituted and/or added in the amino acid sequence set forth in SEQ ID NO: 8, and said sequence has UDP-4-keto-6-deoxyglucose 3,5-epimerase and UDP-4-keto-rhamnose 4-keto-reductase activities;

(12) A DNA, which encodes the protein as described in the above item (11);

(13) A DNA comprising:

(a) a nucleotide sequence represented by SEQ ID NO: 7, or

(b) a nucleotide sequence represented by SEQ ID NO: 7, wherein one or more nucleotides are deleted, substituted and/or added in the nucleotide sequence set forth in SEQ ID NO: 7, and said sequence encodes a protein having UDP-4-keto-6-deoxyglucose 3,5-epimerase and UDP-4-keto-rhamnose 4-keto-reductase activities;

(14) A recombinant vector comprising the DNA as described in the above item (12) or (13);

(15) A transformant, which is obtained by introducing the recombinant vector as described in the above item (14) into a host cell;

(16) A recombinant vector comprising the DNA as described in the above item (7) or (8) and the DNA as described in the above item (12) or (13);

(17) A transformant, which is obtained by introducing the recombinant vector as described in the above item (9) and the recombinant vector as described in the above item (14) into the same host cell;

(18) A transformant, which is obtained by introducing the recombinant vector as described in the above item (16) into a host cell;

(19) A protein, comprising an amino acid sequence with 70% or more homology with an amino acid sequence represented by SEQ ID NO: 2, SEQ ID NO: 4 or SEQ ID NO: 20,

wherein said sequence has UDP-glucose 4,6-dehydratase, UDP-4-keto-6-deoxyglucose 3,5-epimerase and UDP-4-keto-rhamnose 4-keto-reductase activities;

(20) The protein as described in the above item (19), which is a protein derived from a rice plant (Oryza sativa) or a tobacco plant (Nicotiana tabacum).

(21) A DNA, which encodes the protein as described in the above item (19) or (20);

(22) A recombinant vector comprising the DNA as described in the above item (21);

(23) A transformant, which is obtained by introducing the recombinant vector as described in the above item (22) into a host cell;

(24) The transformant as described in any of the above items (5), (17), (18) and (23), wherein the host cell contains UDP-glucose in the cell;

(25) The transformant as described in any of the above items (5), (17), (18), (23) and (24), wherein the host cell does not have an enzyme for consuming UDP-rhamnose;

(26) The transformant as described in any of the above items (5), (10), (16) to (18), and (23) to (25), wherein the host cell is a yeast cell;

(27) A method of producing UDP-rhamnose, comprising the steps of:

culturing the transformant as described in any one of the above items (5), (17), (18) and (23) to (26), to give a culture, and

extracting UDP-rhamnose from the culture;

(28) A method of producing UDP-rhamnose, comprising the steps of:

culturing the transformant as described in any one of the above items (5), (17), (18) and (23) to (26) in a medium, to give a culture, and

bringing at least one of the resulting culture, a material obtained by treating the culture, an enzyme extract and a purified enzyme into contact with UDP-glucose, thereby converting UDP-glucose into UDP-rhamnose;

(29) A method of producing a labeled UDP-rhamnose, comprising the steps of:

culturing the transformant as described in any one of the above items (5), (17), (18) and (23) to (26) with an isotope-substituted glucose as a carbon source, to give a culture, to give a culture, and

extracting an isotope-labeled UDP-rhamnose from the resulting culture;

(30) A method of producing a labeled UDP-rhamnose, comprising the steps of:

bringing at least one of a culture, a material obtained by treating the culture, an enzyme extract and a purified enzyme, which is obtained by culturing the transformant as described in any one of the above items (5), (17), (18) and (23) to (26) in a medium, into contact with an isotope-substituted UDP-glucose, and

extracting an isotope-labeled UDP-rhamnose; and

(31) The method of producing a labeled UDP-rhamnose as described in the above item (29) or (30), wherein the isotope is C¹³ or C¹⁴.

The present invention is explained in detail below.

The first enzyme protein of the present invention is a protein derived from Arabidopsis (Arabidopsis thaliana), having three (3) activities, that is, UDP-glucose 4,6-dehydratase, UDP-4-keto-6-deoxyglucose 3,5-epimerase and UDP-4-keto-rhamnose 4-keto-reductase activities, and having an amino acid sequence represented by SEQ ID NO: 2, SEQ ID NO: 4 or SEQ ID NO: 20 (hereinafter, the enzyme proteins represented by SEQ ID NO: 2, SEQ ID NO: 4 and SEQ ID NO: 20 are also referred to as RHM1 protein, RHM2 protein and RHM3 protein, respectively). But the first enzyme protein in the present invention is not limited thereto and includes a protein having an amino acid sequence represented by SEQ ID NO: 2, SEQ ID NO: 4 or SEQ ID NO: 20, wherein one or more amino acids are deleted, substituted and/or added in the amino acid sequence set forth in SEQ ID NO: 2, SEQ ID NO: 4 or SEQ ID NO: 20, and said sequence has UDP-glucose 4,6-dehydratase-UDP-4-keto-6-deoxyglucose 3,5-epimerase/UDP-4-keto-rhamnose 4-keto-reductase activities.

The first enzyme gene of the present invention is a DNA encoding the amino acid sequence described above. Specifically, the nucleotide sequence is represented by SEQ ID NO: 1, SEQ ID NO: 3 or SEQ ID NO: 19 (hereinafter, the genes represented by SEQ ID NO: 1, SEQ ID NO: 3 and SEQ ID NO: 19 are also referred to as RHM1 gene, RHM2 gene and RHM3 gene, respectively), but the first enzyme gene in the present invention is not limited thereto and also includes a gene having a nucleotide sequence represented by SEQ ID NO: 1, SEQ ID NO: 3 or SEQ ID NO: 19, wherein one or more nucleotides are deleted, substituted and/or added in the nucleotide sequence set forth in SEQ ID NO: 1, SEQ ID NO: 3 or SEQ ID NO: 19, and said sequence encodes a protein having UDP-glucose 4,6-dehydratase, UDP-4-keto-6-deoxyglucose 3,5-epimerase and UDP-4-keto-rhamnose 4-keto-reductase activities.

In the present invention, the Arabidopsis thaliana RHM1 gene, RHM2 gene, RHM3 gene, or the mutated gene described above can be used to produce UDP-rhamnose efficiently by genetic engineering means, wherein the production method is divided roughly into 2 methods, that is, a method of producing it in cells (culture method) and a method of producing it outside cells (enzyme conversion method).

Among these methods, the method of producing UDP-rhamnose in cells comprising the following steps, for example:

(1) Arabidopsis thaliana RHM1, RHM2 or RHM3 gene [UDP-glucose 4,6-dehydratase, UDP-4-keto-6-deoxyglucose 3,5-epimerase and UDP-4-keto-rhamnose 4-keto-reductase gene (UDP-glucose 4,6-dehydratase-3,5-epimerase/4-keto-reductase gene)] is obtained;

(2) The Arabidopsis thaliana RHM1, RHM2 or RHM3 gene is introduced into a vector, such as expression plasmid, to construct a vector comprising the RHM1, RHM2 or RHM3 gene;

(3) The expression vector is introduced into a host cell having UDP-glucose in the cell, to transform the host cell; and

(4) The recombinant in the above (3) is cultured in a medium; and UDP-rhamnose is collected from the resulting culture or a material obtained by treating the culture such as disrupted cell materials, and further isolated and/or purified.

In the present invention, the method of producing UDP-rhamnose outside cells comprising the following steps:

(1) Arabidopsis thaliana RHM1, RHM2 or RHM3 gene (UDP-glucose 4,6-dehydratase-3,5-epimerase/4-keto-reductase gene) is obtained;

(2) The Arabidopsis thaliana RHM1, RHM2 or RHM3 gene is introduced into a vector, such as expression plasmid, to construct a vector comprising the RHM1, RHM2 or RHM3 gene;

(3) The expression vector is introduced into a host cell having UDP-glucose in the cell, to transform the host cell; and

(4) A culture, a material obtained by treating the culture such as disrupted cell materials, an intracellular extract, or an enzyme isolated and/or purified from the extract, is used as an enzyme source, and UDP-glucose and NADPH are added thereby converting UDP-glucose into UDP-rhamnose which is then isolated and/or purified. The UDP-rhamnose is essential as a glycosyl donor for synthesis of a rhamnose-containing sugar chain and is thus useful for adding rhamnose to a sugar chain considered functionally important.

Alternatively, for example, a culture of the transformant transformed with an expression vector containing Arabidopsis thaliana RHM1, RHM2 or RHM3 gene, a material obtained by treating the culture, an intracellular extract or an enzyme isolated and/or purified from the extract, and a culture of a cell inherently producing UDP-glucose, a material obtained by treating the culture, an intracellular extract or an enzyme isolated and/or purified from the extract are prepared separately, and these materials are allowed to act sequentially or simultaneously on UDP-glucose, to convert UDP-glucose into UDP-rhamnose thereby producing UDP-rhamnose.

The RHM2 protein represented by SEQ ID NO: 4 and its gene include, in their sequence, an UDP-glucose 4,6-dehydratase domain, an UDP4-keto-6-deoxyglucose 3,5-epimerase domain and an UDP-4-keto-rhamnose 4-keto-reductase domain.

The second enzyme protein of the present invention is a protein corresponding to the UDP-glucose 4,6-dehydratase domain of the RHM2 protein, which has an amino acid sequence represented by SEQ ID NO: 6, or an amino acid sequence represented by SEQ ID NO: 6, wherein one or more amino acids are deleted, substituted and/or added in the amino acid sequence set forth in SEQ ID NO: 6, and said sequence has an UDP-glucose 4,6-dehydratase activity (hereinafter, the enzyme protein represented by SEQ ID NO: 6 is also referred to as RHM2-N protein).

The second gene of the present invention is a DNA encoding the RHM2-N protein or its mutated protein. Specifically, the second gene is a gene represented by SEQ ID NO: 5 (hereinafter, the gene represented by SEQ ID NO: 5 is also referred to as RHM2 gene), but the second gene of the present invention is not limited thereto and also includes a nucleotide sequence represented by SEQ ID NO: 5, wherein one or more nucleotides are deleted, substituted and/or added in the nucleotide sequence set forth in SEQ ID NO: 5, and said sequence encodes a protein having enzyme activity, that is, a UDP-glucose 4,6-dehydratase activity.

The third enzyme protein of the present invention is a protein which contains both the UDP-4-keto-6-deoxyglucose 3,5-epimerase domain and UDP-4-keto-rhamnose-4-keto-reductase domain of the RHM2 protein, which has an amino acid sequence represented by SEQ ID NO: 8, or an amino acid sequence represented by SEQ ID NO: 8, wherein one or more amino acids are deleted, substituted and/or added in the amino acid sequence set forth in SEQ ID NO: 8, and said sequence has the 2 enzyme activities of UDP-4-keto-6-deoxyglucose 3,5-epimerase and UDP-4-keto-rhamnose-4-keto-reductase (hereinafter, the enzyme protein represented by SEQ ID NO: 8 is also referred to as RHM2-C protein.).

The third gene of the present invention is a DNA encoding the RHM2-C protein or its mutated protein. Specifically, the third gene is a nucleotide sequence represented by SEQ ID NO: 7 (hereinafter, the gene represented by SEQ ID NO: 7 is also referred to as RHM2-C gene), but the third gene of the present invention is not limited thereto and also includes a nucleotide sequence represented by SEQ ID NO: 7, wherein one or more nucleotides are deleted, substituted and/or added in the nucleotide sequence set forth in SEQ ID NO: 7, and said sequence encodes a protein having the 2 enzyme activities, that is, the UDP-4-keto-6-deoxyglucose 3,5-epimerase activity and the UDP-4-keto-rhamnose 4-keto-reductase activity.

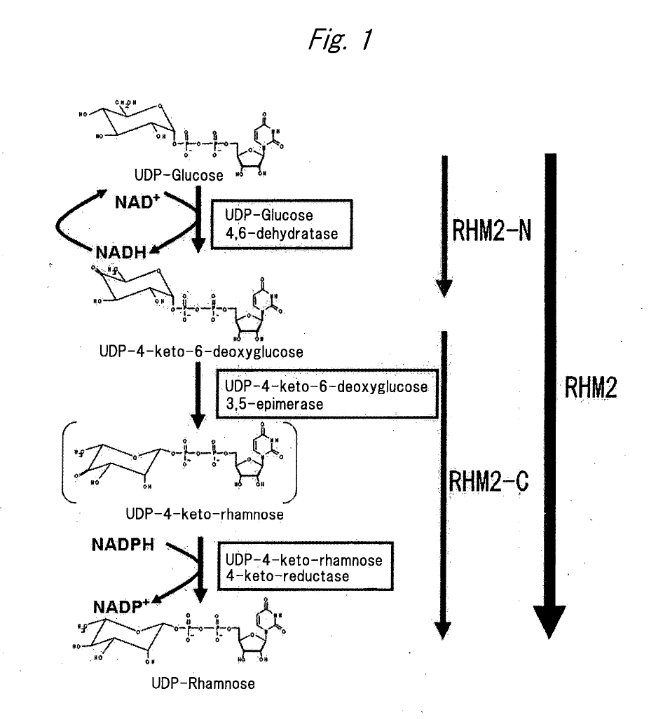

The second and third enzyme proteins can be obtained easily by usual genetic engineering techniques using the second and third genes. The resulting second and third enzyme proteins, as shown in FIG. 1, catalyze a reaction of converting UDP-glucose into UDP-4-keto-deoxyglucose and a reaction of converting UDP-4-keto-deoxyglucose into UDP-rhamnose, respectively, and thus these enzymes can be used sequentially or simultaneously to produce UDP-rhamnose from UDP-glucose.

However, the most productive method of production of UDP-rhamnose in the present invention is a method of co-expressing the second and third genes in the same host cell. The amount of UDP-rhamnose produced is significantly improved by this method as compared with the method of using the RHM2 gene.

Hereinafter, the present invention is described in more detail. Amino acid sequences and nucleotide sequences in this specification, when indicated by abbreviation, shall be in accordance with the IUPAC-IUB regulations or with common names or common practice in the art.

The RHM1, RHM2 or RHM3 gene, RHM2-N gene and RHM2-C gene of the present invention can be obtained by isolation after the PCR method wherein a cDNA library prepared in a usual manner from Arabidopsis thaliana is used as a template. For PCR of the RHM2-N gene and RHM2-C gene, the RHM2 gene may be used as a template.

A cDNA library of Arabidopsis thaliana can be prepared according to a usual method using a usually used plasmid vector or λ-phage-derived vector.

The PCR method is a technique in which a special region of DNA can be amplified specifically 100,000- to 1,000,000-fold over about 2 to 3 hours in vitro with a combination of its sense and antisense primers, thermostable DNA polymerase, and DNA amplification system, and the gene of the present invention and its fragment can be amplified by this PCR method on the basis of homology of the nucleotide sequence with that for the enzyme from another species, or the like.

The vector in which the above gene is integrated may be any vector capable of replication and retention in a host. Examples of the vector include E. coli-derived plasmid vectors pBR322 and pUC19.

The method of integrating the vector can follow, for example, the method of T. Maniatis et al. [Molecular Cloning, Cold Spring Harbor Laboratory, p. 239 (1982)].

The cloned gene can be linked downstream of a promoter in a vector suitable for expression to give an expression vector. Examples of the vector include yeast-derived plasmids YEp352GAP, YEp51, pSH19, and the like.

The genes may have ATG as translation initiation codon at its 5′-end and may have TAA, TGA or TAG as translation termination codon at its 3′-end. The genes may have and express a 6× histidine sequence, a gene for labeling antigen as a part of hemagglutinin protein, or a gene for labeling protein such as GST protein at its 5′- or 3′-end, to facilitate isolation and/or purification of the protein. Particularly, the termination codon is located preferably at the C-terminal of RHM2-N gene represented by SEQ ID NO: 5, and the initiation codon (ATG) is located preferably at the N-terminal of RHM2-C gene represented by SEQ ID NO: 7, and the resulting proteins naturally each have the enzyme activity even without methionine. On the other hand, for example, the proteins having a Met-His tag added to the N-terminal also each have the activity.

For expressing the genes, a promoter is located upstream of the genes, and the promoter that can be used in the present invention may be any suitable promoter compatible with the host used in expression of the genes.

When the host to be transformed is a yeast, examples of the promoter used include ENO1 promoter, GAL10 promoter, GAPDH promoter, ADH promoter, and AOX promoter.

The thus-constructed vector comprising a recombinant DNA having the RHM1, RHM2 or RHM3 gene, RHM2-N gene or RHM2-C gene is used to produce a transformant containing the vector. When the RHM2-N gene and RHM2-C gene are co-expressed, for example, a recombinant vector having the RHM2-N gene and a recombinant vector having the RHM2-C gene are introduced into the same host cell; alternatively, both the RHM2-N gene and RHM2-C gene are introduced into the same vector, and the resulting recombinant vector is used to transform a host cell. The transformant thus obtained harbors 2 kinds of recombinant vectors, that is, the recombinant vector having the RHM2-N gene and the recombinant vector having the RHM2-C gene, or harbors one kind of recombinant vector having both the RHM2-N gene and RHM2-C gene, to produce UDP-glucose 4,6-dehydratase, UDP-4-keto-6-deoxyglucose 3,5-epimerase and UDP-4-keto-rhamnose 4-keto-reductase. Accordingly, the transformant is a highly productive strain capable of producing UDP-rhamnose from UDP-glucose as the starting material, as described above.

Examples of the host includes budding yeast (Saccharomyces cerevisia) and the like not consuming UDP-rhamnose in the living body, and may also be another yeast (Pichia pastoris etc.) not consuming UDP-rhamnose. The host may also be a non-yeast host not having an enzyme system for consuming UDP-rhamnose.

When UDP-rhamnose is produced in vitro by using the disrupted cell material, the enzyme extract or the like, any host capable of producing the gene product in the cytoplasm can be used.

Formation of the transformant is carried out by a method generally carried out for the intended host. The method may not be a general method insofar as the method is applicable. For example, when the host is yeast, a vector containing the recombinant DNA can be introduced by the lithium method or electroporation method.

In this manner, a transformant containing a vector containing the recombinant DNA having the gene of the present invention can be obtained, and the transformant is cultured thereby producing and accumulating UDP-rhamnose and the enzymes necessary for production of UDP-rhamnose, mainly in cells of the transformant.

When the transformant is cultured, the medium used in the culturing is a general medium used for the intended host. The medium may not be a general medium if it is applicable. For example, when the host is a yeast, YPD medium, SD medium or the like is generally used. The culture is carried out under conditions generally used for the intended host. The conditions may not be general insofar as the conditions are applicable. By way of example, when the host is a yeast, the culture can be carried out at about 25 to 37° C. for about 12 hours to 5 days, if necessary under aeration or stirring.

For example, when UDP-rhamnose or the enzyme is extracted from the culture, host cells are separated from the medium by centrifugation, and the host cells are disrupted to extract UDP-rhamnose or the enzyme. When the host is a yeast, for example, the cells are disrupted with glass beads and then centrifuged. In this case, the enzyme and UDP-rhamnose are present in the supernatant fraction. Alternatively, the yeasts may be suspended in 1 M formic acid saturated with 1-butanol, then cooled on ice for about 30 minutes to 3 hours and centrifuged to extract UDP-rhamnose. In this case too, UDP-rhamnose is present in the supernatant fraction.

When UDP-rhamnose is separated from the supernatant fraction separated by centrifugation, low-molecular-weight fractions are collected by gel filtration or the like and further separated by HPLC, to purify and/or isolate UDP-rhamnose. Alternatively, UDP-rhamnose can be purified and/or isolated through an ion-exchange column or a reverse-phase column.

When UDP-rhamnose is produced by enzyme conversion reaction in vitro, the disrupted cell material or the supernatant fraction can be used directly as the enzyme source; alternatively, after ammonium sulfate is dissolved at a concentration of 75% in the supernatant fraction, a protein fraction precipitated with ammonium sulfate is collected and desalted by dialysis or the like, and the resultant can also be used as the enzyme source.

UDP-glucose as the substrate, NAD⁺ and NADPH are added to the enzyme source and reacted thereof, and thus UDP-rhamnose can be isolated and/or purified by HPLC to give UDP-rhamnose.

In the present invention, on the other hand, not only Arabidopsis thaliana-derived proteins (RHM1 protein, RHM2 protein and RHM3 protein) represented by SEQ ID NO: 2, SEQ ID NO: 4 and SEQ ID NO: 20, but also proteins having 70% or more homology with the RHM1 protein, RHM2 protein or RHM3 protein can be included as the proteins having UDP-glucose 4,6-dehydratase, UDP-4-keto-6-deoxyglucose 3,5-epimerase and UDP-4-keto-rhamnose 4-keto-reductase activities, and the present invention includes these homologous proteins and genes having nucleotide sequences encoding the homologous proteins. These homologous proteins include proteins derived from plants such as a rice plant (Oryza Sativa) or a tobacco plant (Nicotiana tabacum), and the amino acid sequence of the homologous protein from the rice plant and the nucleotide sequence of the gene encoding the same are represented by SEQ ID NOS: 22 and 21, respectively.

These homologous proteins can be produced by using DNAs encoding them with the same genetic engineering means as described with respect to the RHM1 protein, RHM2 protein and RHM3 protein. That is, the present invention also includes a recombinant vector recombined with a DNA encoding the homologous protein, a transformant having the recombinant vector introduced into it, a method of producing the homologous enzyme protein by using the transformant, and a method of producing UDP-rhamnose by using the transformant or the homologous enzyme protein.

In culturing the transformant of the present invention in a medium, the transformant is cultured in a medium containing, as a carbon source, glucose whose element is replaced by an isotope, whereby an isotope-labeled UDP-rhamnose can be collected from the culture.

Alternatively, an enzyme protein-containing culture, a material obtained by treating the culture, an enzyme extract or a purified enzyme, obtained from the transformant of the present invention, can be contacted with UDP-glucose whose element is replaced by an isotope, thereby converting the UDP-glucose into an isotope-labeled UDP-rhamnose.

In such enzyme conversion methods, the RHM2-N purified protein, a culture containing the protein, a material obtained by treating the culture, an enzyme extract or a purified enzyme, and the RHM2-C purified protein, a culture containing the protein, a material obtained by treating the culture or an enzyme extract may be contacted sequentially or simultaneously with the above isotope-containing UDP-glucose, to give an isotope-labeled UDP-rhamnose.

Preferable examples of the isotope include C¹³ or C¹⁴ isotopes. The UDP-rhamnose labeled with such isotope is useful for elucidation of sugar chain synthesis system or for searching a substance exerting influence on the sugar chain synthesis system.

According to the present invention, UDP-rhamnose essential for adding rhamnose having very important functions in a sugar chain can be produced efficiently in a large amount. At this time, techniques of synthesizing glycoprotein sugar chains uniformly is not established, and it is anticipated that synthesis of uniform sugar chains can be carried out by in vitro modification of sugar chains finally, and in this case, sugar nucleotides are essential as the glycosyl donor. With respect to rhamnose, UDP-rhamnose is particularly very expensive, and thus the in vitro modification reaction in a large amount is unrealistic at present, but by enabling the supply of a large amount of UDP-rhamnose according to the present invention, highly functional sugar chains to which rhamnose is added can be carried out in vitro. Accordingly, the present invention contributes significantly to study of sugar chains in glycoproteins.

The present invention will be described in more detail based on the following examples, but the invention is not intended to be limited thereto.

### EXAMPLES

### Example 1

### Isolation of RHM1 Gene (SEQ ID NO: 1), RHM2 Gene (SEQ ID NO: 3) and RHM3 Gene (SEQ ID NO: 19)

The RHM1 gene and RHM2 gene were cloned by the PCR method where a Arabidopsis thaliana cDNA library was used as a template. As the cDNA library, QUICK-Clone cDNA manufactured by CLONTECH was used. With respect to the RHM3 gene, cDNA clone was obtained (Resource Number: pda08705) from BioResource Center, RIKEN, Japan, and used as a template. The primers were designed such that for facilitating cleavage of the portions encoding the protein with restriction enzymes, Sac I site was located in the N-terminal and Kpn I site was located in the C-terminal in the case of RHM1, while Eco RI site was located in the N-terminal and Sal I site was located in C-terminal in the case of RHM2 and RHM3. In addition, a 6× histidine sequence encoding a nickel agarose binding sequence, that is, a labeling antigen, was previously added to the N-terminal. Sequences of the respective primers are shown below (SEQ ID NOS: 13 and 14 were for RHM1; SEQ ID NOS: 15 and 16 were for RHM2; and SEQ ID NOS: 23 and 24 were for RHM3).

(SEQ ID NO: 13)

AAGAGCTCATGCATCACCATCACCATCACATGGCTTCGTACACTCCCAAG
AAC

(SEQ ID NO: 14)

AAAGGTACCTCAGGTTTTCTTGTTTGGCCCGTATG

(SEQ ID NO: 15)

AGAATTCATGCATCACCATCACCATCACATGGATGATACTACGTATAAGC
CAAAGAAC

(SEQ ID NO: 16)

AAAAAGTCGACTTAGGTTCTCTTGTTTGGTTCAAAGAC

(SEQ ID NO: 23)

AGAATTCATGCATCACCATCACCATCACATGGCTACATATAAGCCTAAGA
ACATCCTC

(SEQ ID NO: 24)

AAAAAGTCGACTTACGTTCTCTTGTTAGGTTCGAAGACG

PCR conditions are as follows:

95° C. 30 sec

57° C. 30 sec

72° C. 2 min 15 sec

30 cycle

About 2.0-kbp DNA amplification fragments obtained under these conditions were isolated by agarose electrophoresis and then cleaved with restriction enzymes Sac I and Kpn I or Eco RI and Sal I and then inserted between the Sac I site and the Kpn I site or the Eco RI site and the Sal I site of pBluescript II-SK+ vector. The nucleotide sequences of these cloned genes were confirmed with a sequence kit using a dideoxy method and confirmed to be nucleotide sequences represented by SEQ ID NOS: 1, 3 and 19.

### Example 2

### Construction of RHM1, RHM2 and RHM3 Gene Expression Vectors and Creation of Yeast Transformants Containing the Plasmids

The RHM1 gene, RHM2 gene and RHM3 gene inserted into the pBluescript II-SK+ vector were cleaved off with Sac I and Kpn I or with Eco RI and Sal I, and then inserted between the Sac I site and the Kpn I site or the Eco RI site and the Sal I site in expression vector YEp352GAP-II having URA3 gene as a selection marker and a region of from Eco RI to Sal I in pUC18 multi-cloning site between GAPDH that was a promoter in yeast glycolytic system and its terminator, thereby constructing YEp352-GAP-II-6×HIS-RHM1, YEp352-GAP-II-6×HIS-RHM2 and YEp352-GAP-II-6×HIS-RHM3. These expression vectors were introduced into yeast W303-1B strain (ura3, lue2, his3, trp1, ade2) to give transformants, that is, the RHM1 gene-introduced strain (W303/YEp352-GAP-II-6×HIS-RHM1 strain), the RHM2 gene-introduced strain (W303/YEp352-GAP-II-6×HIS-RHM2 strain) and the RHM3 gene-introduced strain (W303/YEp352-GAP-II-6×HIS-RHM3 strain). The W303-1B strain (ATCC Number: 201238™) was available from ATCC (American Type Culture Collection).

### Example 3

### Extraction of Yeast Cytoplasmic Protein

Cytoplasmic proteins were extracted from the transformants obtained in Example 2. First, the W303/YEp352-GAP-II-6×HIS-RHM1 strain, the W303/YEp352-GAP-II-6×HIS-RHM2 strain, the W303/YEp352-GAP-II-6×HIS-RHM3 strain and the W303 strain were cultured in SD medium for 24 hours at 30° C., and the resulting yeast cells were disrupted with glass beads. The resulting disrupted materials were centrifuged to remove a cell wall fraction and a microsome fraction, whereby a cytoplasmic fraction was separated.

### Example 4

### Confirmation of Expression of RHM1 Protein, RHM2 Protein and RHM3 Protein in Yeasts

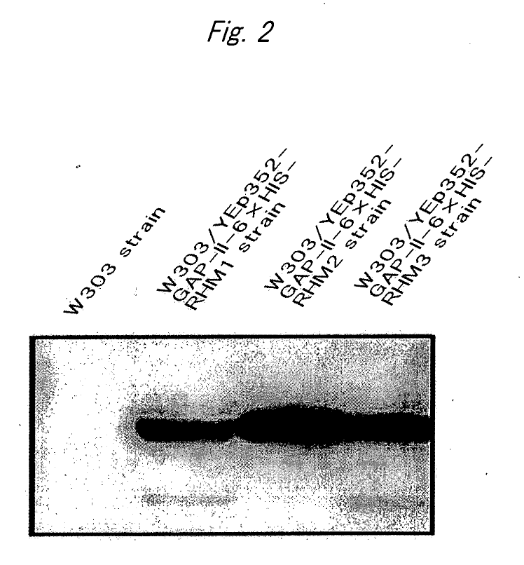

The transformants obtained in Example 2 were confirmed by Western blotting to express the proteins in their cells. First, the above 3 kinds of transformants, that is, the W303/YEp352-GAP-II-6×HIS-RHM1 strain, the W303/YEp352-GAP-II-6×HIS-RHM2 strain and the W303/YEp352-GAP-II-6×HIS-RHM3 strain, and W303 strain, were cultured in SD medium for 24 hours at 30° C., and the resulting yeast cells were disrupted with glass beads. The resulting disrupted materials were centrifuged to remove a cell wall fraction and a microsome fraction, whereby a cytoplasmic fraction was separated. This protein-containing cytoplasmic fraction was dissolved in 50 mM Tris-HCl, pH6.8, then subjected to SDS-PAGE and transferred electrically onto a PVDF membrane. Expression of the proteins was confirmed with anti-penta-HIS antibody (FIG. 2). As a result, it was confirmed that the proteins were expressed in the RHM1 gene-introduced strain [W303/YEp352-GAP-II-6×HIS-RHM1 strain], the RHM2 gene-introduced strain [W303/YEp352-GAP-II-6×HIS-RHM2 strain] and the RHM3 gene-introduced strain [W303/YEp352-GAP-II-6×HIS-RHM3 strain], respectively.

### Example 5

### Measurement of UDP-Rhamnose Synthesis Activity

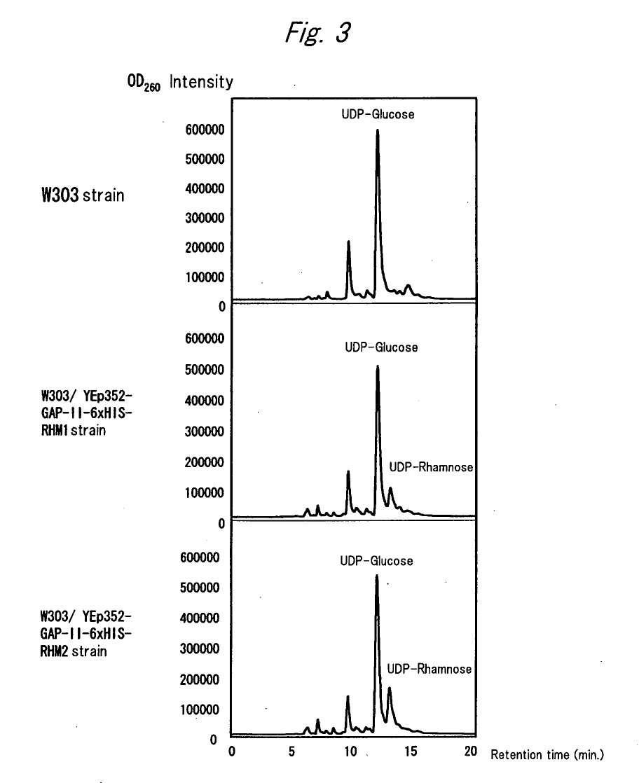

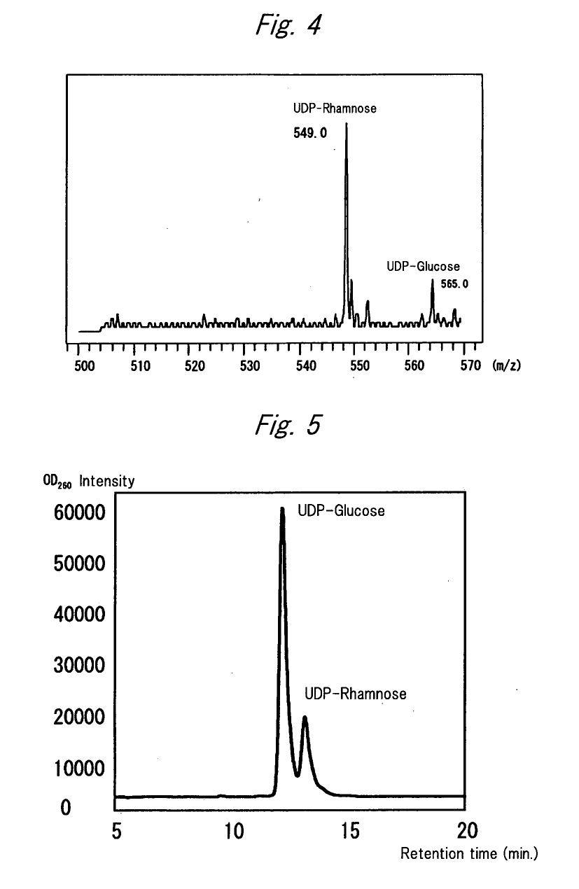

The UDP-rhamnose synthesis activity of the yeast cytoplasmic protein fraction obtained in Example 3 was detected. The activity was measured at 30° C. for 2 hours by using UDP-glucose as a substrate and 1 mM NAD⁺ and 3 mM NADPH as cofactors under the conditions of 100 mM Tris-HCl, pH 8.0. After a phenol/chloroform (24/1) mixture was added, the mixture was stirred and centrifuged to recover a supernatant. The supernatant was applied to HPLC equipped with a revere-phase column to detect UDP-rhamnose and UDP-glucose. The sample was separated with 20 mM triethylamine (pH 7.0) containing 1% acetonitrile passing at a flow rate of 0.7 ml/min through C30 column in HPLC and detected at UV₂₆₀ nm. As a result, UDP-rhamnose synthesis activity was detected in the W303/YEp352-GAP-II-6×HIS-RHM1 strain expressing RHM1 protein, the W303/YEp352-GAP-II-6×HIS-RHM2 strain expressing RHM2 protein, and the W303/YEp352-GAP-II-6×HIS-RHM3 strain expressing RHM3 protein (FIG. 3). The molecular weight of the synthesized UDP-rhamnose, as determined with ESI-MS, agreed with the predicted molecular weight (FIG. 4).

### Example 6

### Extraction of UDP-Rhamnose from Yeasts into which RHM2 Gene was Introduced

From the yeast cytoplasmic fraction obtained in Example 3, UDP-rhamnose was separated and purified. First, the W303/YEp352-GAP-II-6×HIS-RHM2 strain was cultured for 24 hours at 30° C. in SD (uracil⁻) medium, and then 1 ml of 1 M formic acid saturated with 1-butanol was added to the yeast cells per O.D.600=10, and the mixture was sufficiently mixed and left on ice for 1 hour. A cell wall fraction was removed from this sample by centrifugation, whereby the cytoplasmic fraction was separated. The thus-obtained supernatant was lyophilized and re-suspended in purified water, to extract sugar nucleotides. The extracted sugar nucleotides were separated with 20 mM triethylamine (pH 7.0) containing 1% acetonitrile passing at a flow rate of 0.7 ml/min through C30 column, to separate UDP-rhamnose on the basis of elution time. The separated UDP-rhamnose was re-separated several times under the same conditions, whereby UDP-rhamnose was purified (FIG. 5).

### Example 7

### Expression of UDP-Glucose 4,6-Dehydratase Domain and UDP-4-Keto-6-Deoxyglucose 3,5-Epimerase/UDP-4-Keto-Rhamnose 4-Keto-Reductase Domain by RHM2 Gene

The UDP-glucose 4,6-dehydratase domain (RHM2-N) and the UDP-4-keto-6-deoxyglucose 3,5-epimerase/UDP-4-keto-rhamnose 4-keto-reductase domain (RHM2-C) in the RHM2 gene were cloned by the PCR method using YEp352-GAP-II-6×HIS-RHM2 obtained in Example 2. The primers were designed such that Eco RI site was located in the N-terminal and Sal I site in the C-terminal in both RHM2-N and RHM2-C domains in order to facilitate cleavage of the portions encoding the proteins with restriction enzymes. In addition, a 6× histidine sequence encoding a nickel agarose binding sequence, that is, a labeling antigen, was previously added to the N-terminal. The primers were SEQ ID NOS: 15 and 16 used in Example 1. Sequences of the other primers are shown below (SEQ ID NOS: 15 and 17 were for RHM2-N, and SEQ ID NOS: 16 and 18 were for RHM2-C).

(SEQ ID NO: 17)

AAAAAGTCGACTTAAGCTTTGTCACCAGAATCACCATT

(SEQ ID NO: 18)

AGAATTCATGCATCACCATCACCATCACACACCTAAGAATGGTGATTCTG
GTG

PCR conditions are as follows:

95° C. 30 sec

57° C. 30 sec

72° C. 1 min 15 sec

30 cycle

About 1.2-kb (RHM2-N) and about 0.9-kb (RHM2-C) fragments obtained under these conditions were isolated by agarose electrophoresis, then cleaved with restriction enzymes Eco RI and Sal I, inserted between the Eco RI and Sal I sites of YEp352GAP-II, to construct YEp352-GAP-II-6×HIS-RHM2-N and YEp352-GAP-II-6×HIS-RHM2-C. The nucleotide sequences of these cloned genes were confirmed by nucleotide sequence analysis using a dideoxy method and confirmed to be nucleotide sequences represented by SEQ ID NOS: 9 and 11, respectively (amino acid sequences of their corresponding proteins are shown in SEQ ID NOS: 10 and 12, respectively). From the YEp352-GAP-II-6×HIS-RHM2-C vector, a fragment containing a sequence of 3 regions, e.g., GAPDH promoter, 6×HIS-RHM2-C, and GAPDH terminator, was cleaved off with restriction enzyme Bam HI and inserted between the Bam HI sites of yeast plasmid vector YEp351 having LEU2 marker, to construct YEp351-GAP-II-6×HIS-RHM2-C. These expression vectors were introduced into yeast W303-1B strain (ura3, lue2, his3, trp1, ade2) to give transformants, e.g., the RHM2-N gene-introduced strain (W303/YEp351-GAP-II-6×HIS-RHM2-N strain), the RHM2-C gene-introduced strain (W303/YEp351-GAP-II-6×HIS-RHM2-C strain), and the RHM2-N+RHM2-C strain into which the RHM2-N gene and RHM2-C gene simultaneously introduced (W303/YEp351-GAP-II-6×HIS-RHM2-N, YEp351-GAP-II-6×HIS-RHM2-C strain).

### Example 8

### Extraction of Yeast Cytoplasmic Proteins

From the transformants (the RHM2-N strain, RHM2-C strain, and RHM2-N+RHM2-C strain) obtained in Example 7, cytoplasmic proteins were extracted according to the method shown in Example 3. First, these transformants and the W303 strain were cultured in SD medium for 24 hours at 30° C., and the resulting yeast cells were disrupted with glass beads. The resulting disrupted materials were centrifuged to remove a cell wall fraction and a microsome fraction, whereby a cytoplasmic fraction was separated.

### Example 9

### Confirmation of Expression of RHM2-N Protein and RHM2-C Protein in Yeasts

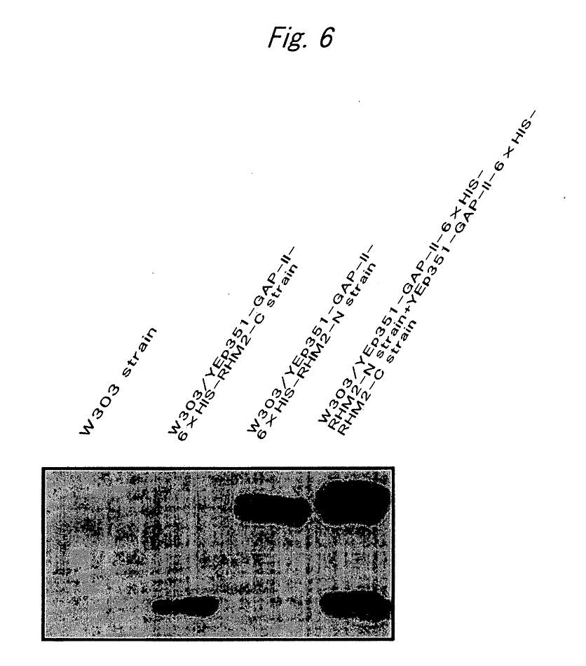

The transformants obtained in Example 7 were confirmed by Western blotting to express the proteins in their cells. As the Western blotting samples, the yeast cytoplasmic proteins extracted in Example 8 were used. The protein-containing cytoplasmic fraction was subjected to SDS-PAGE and then transferred electrically onto a PVDF membrane, and expression of the proteins was confirmed by anti-penta-HIS antibody (FIG. 6). As a result, it was confirmed that the proteins were expressed in the RHM2-N gene-introduced strain (W303/YEp351-GAP-II-6×HIS-RHM2-N strain), the RHM2-C gene-introduced strain (W303/YEp351-GAP-II-6×HIS-RHM2-C strain), and the RHM2-N+RHM2-C strain into which the RHM2-N gene and RHM2-C gene simultaneously introduced (W303/YEp351-GAP-II-6×HIS-RHM2-N, YEp351-GAP-II-6×HIS-RHM2-C strain), respectively.

### Example 10

### Measurement of UDP-Rhamnose Synthesis Activity in RHM2-N+C Strain (W303/YEp351-GAP-II-6×HIS-RHM2-N, YEp351-GAP-II-6×HIS-RHM2-C Strain)

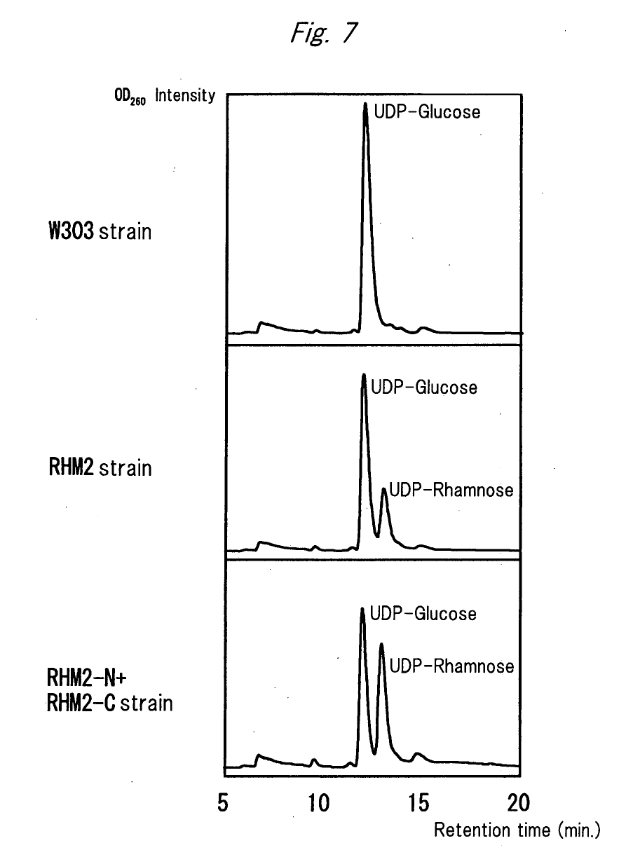

The UDP-rhamnose synthesis activity in the yeast cytoplasmic protein fraction obtained in Example 8 was detected. Simultaneously, the UDP-rhamnose synthesis activities in the protein fraction extracted from W303 strain and in the protein fraction extracted from the W303/YEp352-GAP-II-6×HIS-RHM2 strain obtained in Example 3, were also detected. The activities were measured at 37° C. for 3 hours with UDP-glucose as a substrate and 1 mM NAD⁺ and 3 mM NADPH as cofactors under the condition of 100 mM Tris-HCl, pH 7.8. After a mixture of phenol/chloroform (24/1) mixture was added, the mixture was stirred and centrifuged to recover a supernatant. The supernatant was applied to HPLC equipped with a reverse-phase column to detect UDP-rhamnose and UDP-glucose. The sample was separated with 20 mM triethylamine (pH 7.0) containing 1% acetonitrile passing at a flow rate of 0.7 ml/min through C30 column in HPLC and detected at UV₂₆₀ nm. As a result, the RHM2-N+RHM2-C strain (W303/YEp351-GAP-II-6×HIS-RHM2-N, YEp351-GAP-II-6×HIS-RHM2-C strain) showed higher efficiency of synthesis of UDP-rhamnose than that by the W303/YEp352-GAP-II-6×HIS-RHM2 strain (FIG. 7).

Having described our invention as related to the present embodiments, it is our intention that the invention not be limited by any of the details of the description, unless otherwise specified, but rather be construed broadly within its spirit and scope as set out in the accompanying claims.

This non-provisional application claims priority under 35 U.S.C. § 119 (a) on Patent Application No. 2006-142359 filed in Japan on May 23, 2006, and Patent Application No. 2006-204421 filed in Japan on Jul. 27, 2006, each of which is entirely herein incorporated by reference.

What we claim is:

1. A protein comprising:
(a) an amino acid sequence represented by SEQ ID NO: 2, SEQ ID NO: 4 or SEQ ID NO: 20, or
(b) an amino acid sequence represented by SEQ ID NO: 2, SEQ ID NO: 4 or SEQ ID NO: 20, wherein one or more amino acids are deleted, substituted and/or added in the amino acid sequence set forth in SEQ ID NO: 2, SEQ ID NO: 4 or SEQ ID NO: 20, and said sequence has UDP-glucose 4,6-dehydratase, UDP-4-keto-6-deoxyglucose 3,5-epimerase and UDP-4-keto-rhamnose 4-keto-reductase activities.

2. A DNA, which encodes the protein according to claim 1.

3. A DNA comprising:
(a) a nucleotide sequence represented by SEQ ID NO: 1, SEQ ID NO: 3 or SEQ ID NO: 19, or
(b) a nucleotide sequence represented by SEQ ID NO: 1, SEQ ID NO: 3 or SEQ ID NO: 19, wherein one or more nucleotides are deleted, substituted and/or added in the nucleotide sequence set forth in SEQ ID NO: 1, SEQ ID NO: 3 or SEQ ID NO: 19, and said sequence encodes a protein having UDP-glucose 4,6-dehydratase, UDP-4-keto-6-deoxyglucose 3,5-epimerase and UDP-4-keto-rhamnose 4-keto-reductase activities.

4. A recombinant vector comprising the DNA according to claim 3.

5. A transformant, which is obtained by introducing the recombinant vector according to claim 4 into a host cell.

6. A protein comprising:
(a) an amino acid sequence represented by SEQ ID NO: 6, or
(b) an amino acid sequence represented by SEQ ID NO: 6, wherein one or more amino acids are deleted, substituted and/or added in the amino acid sequence set forth in SEQ ID NO: 6, and said sequence has a UDP-glucose 4,6-dehydratase activity.

7. A DNA, which encodes the protein according to claim 6.

8. A DNA comprising:
(a) a nucleotide sequence represented by SEQ ID NO: 5, or
(b) a nucleotide sequence represented by SEQ ID NO: 5, wherein one or more nucleotides are deleted, substituted and/or added in the nucleotide sequence set forth in SEQ ID NO: 5, and said sequence encodes a protein having a UDP-glucose 4,6-dehydratase activity.

9. A recombinant vector comprising the DNA according to claim 8.

10. A transformant, which is obtained by introducing the recombinant vector according to claim 9 into a host cell.

11. A protein comprising:
(a) an amino acid sequence represented by SEQ ID NO: 8, or
(b) an amino acid sequence represented by SEQ ID NO: 8, wherein one or more amino acids are deleted, substituted and/or added in the amino acid sequence set forth in SEQ ID NO: 8, and said sequence has UDP-4-keto-6-deoxyglucose 3,5-epimerase and UDP-4-keto-rhamnose 4-keto-reductase activities.

12. A DNA, which encodes the protein according to claim 11.

13. A DNA comprising:
(a) a nucleotide sequence represented by SEQ ID NO: 7, or
(b) a nucleotide sequence represented by SEQ ID NO: 7, wherein one or more nucleotides are deleted, substituted and/or added in the nucleotide sequence set forth in SEQ ID NO: 7, and said sequence encodes a protein having UDP-4-keto-6-deoxyglucose 3,5-epimerase and UDP-4-keto-rhamnose 4-keto-reductase activities.

14. A recombinant vector comprising the DNA according to claim 13.

15. A transformant, which is obtained by introducing the recombinant vector according to claim 14 into a host cell.

16. A recombinant vector comprising:
a DNA, comprising (a1) a nucleotide sequence represented by SEQ ID NO: 5, or (b1) a nucleotide sequence represented by SEQ ID NO: 5, wherein one or more nucleotides are deleted, substituted and/or added in the nucleotide sequence set forth in SEQ ID NO: 5, and said sequence encodes a protein having a UDP-glucose 4,6-dehydratase activity, and
a DNA, comprising (a2) a nucleotide sequence represented by SEQ ID NO: 7, or (b2) a nucleotide sequence represented by SEQ ID NO.: 7, wherein one or more nucleotides are deleted, substituted and/or added in the nucleotide sequence set forth in SEQ ID NO: 7, and said sequence encodes a protein having UDP-4-keto-6-deoxyglucose 3,5-epimerase and UDP-4-keto-rhamnose 4-keto-reductase activities.

17. A transformant, which is obtained by introducing a recombinant vector comprising a DNA comprising (a1) a nucleotide sequence represented by SEQ ID NO: 5, or (b1) a nucleotide sequence represented by SEQ ID NO: 5, wherein one or more nucleotides are deleted, substituted and/or added in the nucleotide sequence set forth in SEQ ID NO: 5, and said sequence encodes a protein having a UDP-glucose 4,6-dehydratase activity, and a recombinant vector comprising a DNA comprising (a2) a nucleotide sequence represented by SEQ ID NO: 7, or (b2) a nucleotide sequence represented by SEQ ID NO: 7, wherein one or more nucleotides are deleted, substituted and/or added in the nucleotide sequence set forth in SEQ ID NO: 7, and said sequence encodes a protein having UDP-4-keto-6-deoxyglucose 3,5-epimerase and UDP-4-keto-rhamnose 4-keto-reductase activities, into the same host cell.

18. A transformant, which is obtained by introducing the recombinant vector according to claim 16 into a host cell.

19. A protein, comprising an amino acid sequence with 70% or more homology with an amino acid sequence represented by SEQ ID NO: 2, SEQ ID NO: 4 or SEQ ID NO: 20,
wherein said sequence has UDP-glucose 4,6-dehydratase, UDP-4-keto-6-deoxyglucose 3,5-epimerase and UDP-4-keto-rhamnose 4-keto-reductase activities.

20. The protein according to claim 19, which is a protein derived from a rice plant (Oryza sativa) or a tobacco plant (Nicotiana tabacum).

21. A DNA, which encodes the protein according to claim 19.

22. A recombinant vector comprising the DNA according to claim 21.

23. A transformant, which is obtained by introducing the recombinant vector according to claim 22 into a host cell.

24. The transformant according to claim 5, wherein the host cell contains UDP-glucose in the cell.

25. The transformant according to claim 17, wherein the host cell contains UDP-glucose in the cell.

26. The transformant according to claim 18, wherein the host cell contains UDP-glucose in the cell.

27. The transformant according to claim 23, wherein the host cell contains UDP-glucose in the cell.

28. The transformant according to claim 5, wherein the host cell does not have an enzyme for consuming UDP-rhamnose.

29. The transformant according to claim 17, wherein the host cell does not have an enzyme for consuming UDP-rhamnose.

30. The transformant according to claim 18, wherein the host cell does not have an enzyme for consuming UDP-rhamnose.

31. The transformant according to claim 23, wherein the host cell does not have an enzyme for consuming UDP-rhamnose.

32. The transformant according to claim 5, wherein the host cell is a yeast cell.

33. The transformant according to claim 10, wherein the host cell is a yeast cell.

34. The transformant according to claim 15, wherein the host cell is a yeast cell.

35. The transformant according to claim 17, wherein the host cell is a yeast cell.

36. The transformant according to claim 18, wherein the host cell is a yeast cell.

37. The transformant according to claim 23, wherein the host cell is a yeast cell.

38. A method of producing UDP-rhamnose, comprising the steps of:
culturing the transformant according to claim 5, to give a culture, and extracting UDP-rhamnose from the culture.

39. A method of producing UDP-rhamnose, comprising the steps of:
culturing the transformant according to claim 17, to give a culture, and extracting UDP-rhamnose from the culture.

40. A method of producing UDP-rhamnose, comprising the steps of:
culturing the transformant according to claim 18, to give a culture, and extracting UDP-rhamnose from the culture.

41. A method of producing UDP-rhamnose, comprising the steps of:
culturing the transformant according to claim 23, to give a culture, and extracting UDP-rhamnose from the culture.

42. A method of producing UDP-rhamnose, comprising the steps of:
culturing the transformant according to claim 5 in a medium, to give a culture, and
bringing at least one of the resulting culture, a material obtained by treating the culture, an enzyme extract and a purified enzyme into contact with UDP-glucose, thereby converting UDP-glucose into UDP-rhamnose.

43. A method of producing UDP-rhamnose, comprising the steps of:
culturing the transformant according to claim 17 in a medium, to give a culture, and
bringing at least one of the resulting culture, a material obtained by treating the culture, an enzyme extract and a purified enzyme into contact with UDP-glucose, thereby converting UDP-glucose into UDP-rhamnose.

44. A method of producing UDP-rhamnose, comprising the steps of:
culturing the transformant according to claim 18 in a medium, to give a culture, and
bringing at least one of the resulting culture, a material obtained by treating the culture, an enzyme extract and a purified enzyme into contact with UDP-glucose, thereby converting UDP-glucose into UDP-rhamnose.

45. A method of producing UDP-rhamnose, comprising the steps of:
culturing the transformant according to claim 23 in a medium, to give a culture, and
bringing at least one of the resulting culture, a material obtained by treating the culture, an enzyme extract and a purified enzyme into contact with UDP-glucose, thereby converting UDP-glucose into UDP-rhamnose.

46. A method of producing a labeled UDP-rhamnose, comprising the steps of:
culturing the transformant according to claim 5 with an isotope-substituted glucose as a carbon source, to give a culture, and
extracting an isotope-labeled UDP-rhamnose from the resulting culture.

47. A method of producing a labeled UDP-rhamnose, comprising the steps of:
culturing the transformant according to claim 17 with an isotope-substituted glucose as a carbon source, to give a culture, and
extracting an isotope-labeled UDP-rhamnose from the resulting culture.

48. A method of producing a labeled UDP-rhamnose, comprising the steps of:
culturing the transformant according to claim 18 with an isotope-substituted glucose as a carbon source, to give a culture, and
extracting an isotope-labeled UDP-rhamnose from the resulting culture.

49. A method of producing a labeled UDP-rhamnose, comprising the steps of:
culturing the transformant according to claim 23 with an isotope-substituted glucose as a carbon source, to give a culture, and
extracting an isotope-labeled UDP-rhamnose from the resulting culture.

50. A method of producing a labeled UDP-rhamnose, comprising the steps of:
bringing at least one of a culture, a material obtained by treating the culture, an enzyme extract and a purified enzyme, which is obtained by culturing the transformant according to claim 5 in a medium, into contact with an isotope-substituted UDP-glucose, and
extracting an isotope-labeled UDP-rhamnose.

51. A method of producing a labeled UDP-rhamnose, comprising the steps of:
bringing at least one of a culture, a material obtained by treating the culture, an enzyme extract and a purified enzyme, which is obtained by culturing the transformant according to claim 17 in a medium, into contact with an isotope-substituted UDP-glucose, and
extracting an isotope-labeled UDP-rhamnose.

52. A method of producing a labeled UDP-rhamnose, comprising the steps of:
bringing at least one of a culture, a material obtained by treating the culture, an enzyme extract and a purified enzyme, which is obtained by culturing the transformant according to claim 18 in a medium, into contact with an isotope-substituted UDP-glucose, and
extracting an isotope-labeled UDP-rhamnose.

53. A method of producing a labeled UDP-rhamnose, comprising the steps of:
bringing at least one of a culture, a material obtained by treating the culture, an enzyme extract and a purified enzyme, which is obtained by culturing the transformant according to claim 23 in a medium, into contact with an isotope-substituted UDP-glucose, and
extracting an isotope-labeled UDP-rhamnose.

54. The method of producing a labeled UDP-rhamnose according to claim 46, wherein the isotope is C¹³ or C¹⁴.

55. The method of producing a labeled UDP-rhamnose according to claim 47, wherein the isotope is C¹³ or C¹⁴.

56. The method of producing a labeled UDP-rhamnose according to claim 48, wherein the isotope is C¹³ or C¹⁴.

57. The method of producing a labeled UDP-rhamnose according to claim 49, wherein the isotope is C¹³ or C¹⁴.

58. The method of producing a labeled UDP-rhamnose according to claim 50, wherein the isotope is C¹³ or C¹⁴.

59. The method of producing a labeled UDP-rhamnose according to claim 51, wherein the isotope is C¹³ or C¹⁴.

60. The method of producing a labeled UDP-rhamnose according to claim 52, wherein the isotope is C¹³ or C¹⁴.

61. The method of producing a labeled UDP-rhamnose according to claim 53, wherein the isotope is C¹³ or C¹⁴.
